(19) United States
(12) Patent Application Publication
(10) Pub. No.: US 2026/0074049 A1
(43) Pub. Date: Mar. 12, 2026

(54) TAILORED NUTRITIONAL SUPPLEMENTS FOR ATHLETIC PERFORMANCE

(71) Applicant: OptiGenix, Berkeley, CA (US)

(72) Inventors: Gabriel Abbes, Temecula, CA (US); Jai Williams, Nevada City, CA (US); Laura Hix Glickman, Oakland, CA (US)

(73) Assignee: OptiGenix, Berkeley, CA (US)

(21) Appl. No.: 19/243,803

(22) Filed: Jun. 20, 2025

Related U.S. Application Data
(60) Provisional application No. 63/661,782, filed on Jun. 19, 2024.

(51) Int. Cl. G16H 20/60; G06N 20/00; G16H 10/20; G16H 10/40; G16H 10/60; G16H 50/70

(52) U.S. Cl. CPC G16H 20/60; G06N 20/00; G16H 10/20; G16H 10/40; G16H 10/60; G16H 50/70

(57) ABSTRACT

A personalized supplement formulation system integrates biological data, structured qualitative feedback, and machine learning algorithms to generate individualized nutritional protocols. The system receives biological inputs such as genetic single nucleotide polymorphism (SNP) data, blood biomarkers (e.g., ferritin, vitamin D, B12), lipid profiles, and urinary metabolites. A digital user profile is created by organizing these inputs and calculating derived indices relevant to supplementation. Structured qualitative feedback, including dietary restrictions, perceived wellness, and training goals, is normalized and processed alongside the biological data. A trained machine learning engine analyzes combined inputs to output a tailored supplement formulation specifying ingredient selection, dosage, delivery format, and timing. Instructions are transmitted to a manufacturing system capable of producing the custom formulation. The system supports periodic re-evaluation and iteration based on new biological samples or user-reported feedback, enabling dynamic personalization over time and improving efficacy, compliance, and outcome tracking in athletic and wellness domains.

### CROSS-REFERENCE TO RELATED APPLICATIONS

This application is a U.S. Non-Provisional Utility Patent Application entitled, “TAILORED NUTRITIONAL SUPPLEMENTS FOR ATHLETIC PERFORMANCE” which claims priority to U.S. Provisional Ser. No. 63/661,782 , filed on Jun. 19, 2024, the contents of which is hereby fully incorporated by reference.

### FIELD OF THE EMBODIMENTS

The present invention pertains generally to processes and methods of providing personalized healthcare and, more particularly, to the field of personalized nutritional supplements and supplement formulations.

### BACKGROUND OF THE EMBODIMENTS

Nutrients such as vitamins, minerals, fatty acids, amino acids, antioxidants and other types of supplements are key to human health and, more particularly, to athletic performance. Many different supplements are needed for healthy normal functions of the human body, including water-soluble vitamins (e.g., biotin, folates, niacin, pantothenic acid, riboflavin, thiamin, vitamin B6, cobalamine and vitamin C), fat-soluble vitamins (e.g., vitamin A, vitamin D, vitamin E and vitamin K), minerals (e.g., Calcium, Chloride, Magnesium, Phosphorus, Potassium, Sodium and Sulfur) and trace minerals (e.g., Chromium, Copper, Fluoride, Iodine, Iron, Manganese, Molybdenum, Selenium and Zinc), to name a few.

Various methods of selecting nutritional plans tailored to optimize benefits derived from nutrients are known in the art. For example, U.S. Patent Application Publication No. 2013/0261183A1, published Oct. 3, 2013, teaches nutritional compositions and formulations that optimize nutritional contents, and the same reference moreover teaches methods for tailoring such compositions to optimize levels of nutrients that have beneficial effects within specific ranges are provided, along with dietary plans and formulations comprising dietary products that comprise optimized levels of nutrients derived from phytochemicals, antioxidants, vitamins, minerals, lipids, proteins, carbohydrates, probiotics, prebiotics, microorganisms and fiber. Diet plans and modular nutritional packages comprising food and drink items tailored according to consumer patterns typed by diet, age, size, gender, medical conditions, family history, climate and the like are provided.

Similarly, U.S. Patent Application Publication No. 2015/0269865A1, published Sep. 24, 2015, teaches computer-implemented methods for selecting at least one nutritional supplement for a subject. According to at least one aspect of the method taught therein, a computer-implemented method for selecting at least one nutritional supplement for a subject is carried out by a nutritional supplement matching unit programmed to carry out the steps of the method, which comprise: receiving at least one genetic variation of a subject; automatically matching the at least one genetic variation with at least one nutritional supplement using a nutritional supplement correlation database storing a plurality of correlations of genetic variations with nutritional supplements; and generating a signal indicative of the at least one matched nutritional supplement.

U.S. Pat. No. 10,937,538, filed by Landi, discloses integrated, holistic approach to empower older adults to enhance their quality of life and independence through a personalized lifestyle and nutrition program. This is achieved by measuring the physical status of the adults with respect to endurance/functionality. In addition, their nutritional status is assessed. Based on those assessments recommendations are provided with respect to particular exercise programs and nutrients that support the functions of the body. These methods can be implemented as a software program and executed on computer systems. According to some embodiments, these methods include administering bioactive nutrients to a subject according to recommendations to improve the physical parameters of the subject, at least one of the bioactive nutrients administered to the subject according to the recommendations selected from the group consisting of protein, calcium, and vitamin D.

### SUMMARY OF THE EMBODIMENTS

An objective of the present invention is to provide tailored nutritional supplement packages that cater to the unique biological and athletic profiles of individual athletes. In particular, there is a need to provide one or more personalized nutrition recommendations for an individual including, but not limited to an athlete based on advanced biological analysis that incorporates both genetic testing and other biological analysis, including blood metabolism analysis, lipid analysis, and/or urine analysis. In some embodiments, the data-informed supplement recommendation system integrates one or more biological inputs (e.g., biomarkers), performance feedback, and/or structured qualitative data to dynamically optimize athlete supplementation protocol.

Approaches according to various embodiments of the present general inventive concept the aim to maximize athletic performance and health by addressing specific nutritional needs based on genetic and metabolic data. More generally, example embodiments of the present general inventive concept encompass methods and processes that provide personalized nutrition supplementation based on advanced biological analysis that incorporates genetic testing and other biological analysis, including blood metabolism analysis, lipid analysis, and/or urine analysis.

Disclosed herein are various example embodiments of the present general inventive concept. Thus, in some example embodiments of the present general inventive concept, a method for tailoring nutritional supplement formulations to a user profile comprises creating a user profile that includes genetic and biological data; and preparing a supplement formulation on the basis of said user profile, wherein the supplement formulation is tailored to the user profile.

In some embodiments, the genetic and biological data includes at least one type of data selected from the group consisting of genetic testing data, blood metabolic testing data, lipid testing data, and urine analysis data.

Some example embodiments of the present general inventive concept involve leveraging data from biological analysis and demographic data to customize supplement packages for optimal performance enhancement.

Some embodiments further comprise using machine learning as part of the process of tailoring supplement formulations based on a user's genetic and biological data.

Some embodiments comprise iterative development of tailored supplement formulations, continuously refining formulations based on ongoing research and adjusting supplement formulation based on changes in a user's data profile, as well as based on customer feedback.

Further benefits and advantages of the present invention will become apparent after a careful reading of the detailed description.

### BRIEF DESCRIPTION OF THE DRAWINGS

The present disclosure may be better understood, and its numerous features and advantages made apparent to those skilled in the art, by referencing the accompanying drawings. The use of the same reference symbols in different drawings indicates similar or identical items.

FIG. 1 is a flowchart illustrating a computer-implemented method for generating a personalized nutritional supplement formulation for an athletic user, according to some embodiments of the present disclosure.

FIG. 2 is a block diagram illustrating a system, according to some embodiments of the present disclosure.

FIG. 3 is a flowchart illustrating a method, according to some embodiments of the present disclosure.

### DETAILED DESCRIPTION OF THE EMBODIMENTS

Reference will now be made to the example embodiments of the present general inventive concept, examples of which are described below. The example embodiments are described herein in order to explain the present general inventive concept. The present general inventive concept is not limited to any of the examples provided herein. The various embodiments described herein are provided by way of examples, not by way of limitation.

In accordance with the present invention, there is described herein methods and processes that provide personalized nutritional supplement packages tailored to a user's unique profile based on advanced biological analysis that incorporates genetic testing and other biological analysis, including blood metabolism analysis, lipid analysis, and/or urine analysis.

The following detailed description is provided to assist the reader in gaining a comprehensive understanding of the structures and fabrication techniques described herein. Accordingly, various changes, modification, and equivalents of the structures and fabrication techniques described herein will be suggested to those of ordinary skill in the art. The progression of fabrication operations described are merely examples, however, and the sequence type of operations is not limited to that set forth herein and may be changed as is known in the art, with the exception of operations necessarily occurring in a certain order. Also, description of well-known functions and constructions may be simplified and/or omitted for increased clarity and conciseness.

Note that spatially relative terms, such as “up,” “down,” “right,” “left,” “beneath,” “below,” “lower,” “above,” “upper” and the like, may be used herein for ease of description to describe one element or feature's relationship to another element(s) or feature(s) as illustrated in the figures. Spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

Thus, in some example embodiments of the present general inventive concept, a method for tailoring nutritional supplement formulations to a user profile comprises creating a user profile that includes genetic and biological data; and preparing a supplement formulation on the basis of said user profile, wherein the supplement formulation is tailored to the user profile. In some embodiments, the genetic and biological data includes at least one type of data selected from the group consisting of genetic testing data, blood metabolic testing data, lipid testing data, and urine analysis data.

By leveraging advanced biological analysis to tailor supplement packages that cater to the unique biological and athletic profiles of individual athletes, the present general inventive concept aims to maximize athletic performance and health by addressing specific nutritional needs based on genetic and metabolic data. Utilizing cutting-edge genetic testing and blood metabolomic analysis to tailor supplement regimens, personalized nutritional supplement formulations are designed based on confirmed research, focusing on bioavailability and efficacy.

Supplement formulations are not just tailored to individual biological needs but also scientifically validated for absorption and effect. By utilizing a combination of genetic testing, blood metabolomic research, lípidomics, and urine analysis to identify unique nutritional needs, methods and processes according to example embodiments of the present general inventive concept provide bioavailable supplements based on individual data, thereby ensuring efficacy and purity.

Some example embodiments of the present general inventive concept involve leveraging data from biological analysis and demographic data to customize supplement packages for optimal performance enhancement.

Some embodiments further comprise using machine learning to create an automated diagnostic process that registers genetic SNPs, as well as reads demographic data to assess (amongst its available database) the best treatment method for supplementation.

Moreover, some embodiments comprise iterative development of tailored supplement formulations, continuously refining formulations based on ongoing research and adjusting supplement formulation based on changes in a user's data profile, as well as based on customer feedback.

Various example embodiments of the present general inventive concept provide methods and processes that supply personalized nutrition for athletes based on advanced biological analysis that incorporates both genetic testing and other biological analysis, including blood metabolism analysis, lipid analysis, and urine analysis.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

Numerous variations, modification, and additional embodiments are possible, and, accordingly, all such variations, modifications, and embodiments are to be regarded as being within the spirit and scope of the present general inventive concept.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

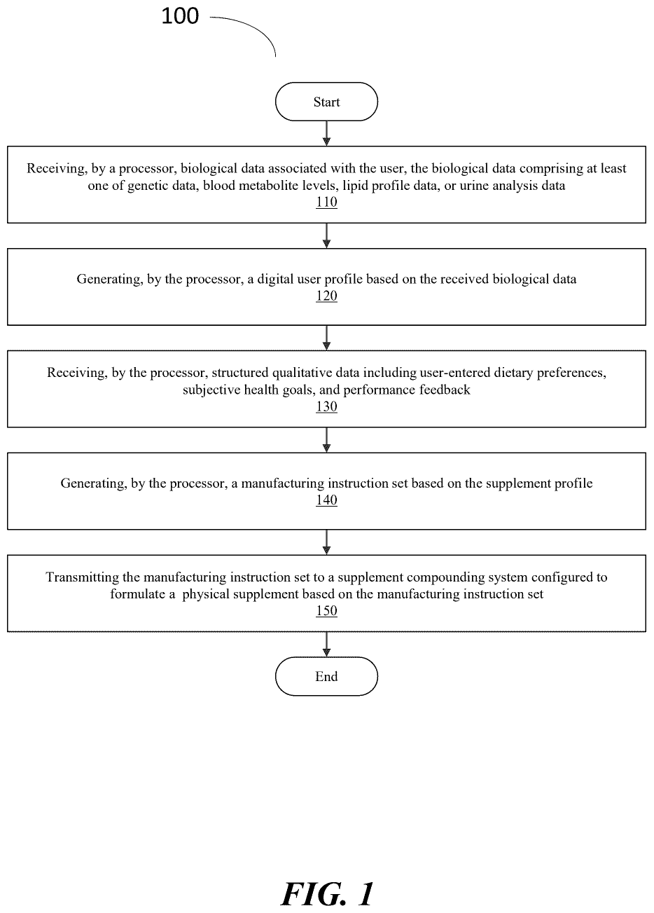

FIGS. 1-3 illustrate example methods and processes for generating and delivering personalized nutritional supplementation protocols based on multi-modal biological analysis, including genetic, metabolic, and physiological inputs. The system integrates biological data collected from genetic sequencing (e.g., SNP panels), blood testing (e.g., vitamin D, B12, ferritin, hs-CRP, Omega-3 Index), lipid profiling (e.g., HDL/LDL cholesterol, triglycerides), and additional samples such as urine and sweat to provide a comprehensive assessment of the user's nutritional and performance status. These data inputs are received through interoperable interfaces with clinical laboratories, wearable devices, and/or at-home test kits.

FIG. 1 illustrates a method 100 for generating a personalized nutritional supplement formulation using a computing system that integrates biological inputs, structured qualitative data, and algorithmic recommendation processes. The method may be implemented on a network-connected computing device or system comprising a processor, memory, and communication interface operable to execute one or more software modules for data ingestion, profile generation, analysis, and output instruction.

At block 110, the method includes receiving, by a processor, biological data associated with the user. The biological data comprises at least one of genetic data (such as genotyping or SNP array data), blood metabolite levels (such as amino acid concentrations, inflammatory markers, or glucose), lipid profile data (e.g., total cholesterol, HDL, LDL, triglycerides), or urine analysis data (e.g., pH, creatinine, or nutrient excretion rates). The biological data may be received through manual entry, file upload, secure API integrations with certified laboratories, or from EHR (electronic health record) systems.

In some embodiments, a step of generating a user profile incorporates both genetic and biological data, forming the foundation for downstream supplement personalization processes. The user profile is a structured digital record that aggregates multimodal inputs collected from the user via an interactive interface. These inputs are obtained through a comprehensive digital intake process involving a guided questionnaire and optional clinical test uploads. The intake process captures over thirty-six discrete data points organized across several physiological, behavioral, and lifestyle categories.

Collected data may include baseline demographic and biometric attributes such as biological sex, age, height, and weight, which are used for population-level stratification and normalization of biological values. The intake also captures physical training characteristics including frequency, intensity, modality (e.g., resistance training, endurance, HIIT), and time-of-day preferences. These variables inform the system's understanding of metabolic load and recovery demands. Additional sections gather user-reported data on recovery dynamics, such as muscle soreness duration and exercise-induced fatigue, as well as sleep quantity and subjective sleep quality, which may reflect recovery status or hormonal imbalances.

Stress level indicators are also collected, either through subjective measures (e.g., perceived stress scales) or behavioral proxies such as variability in sleep or workout compliance. Dietary input includes patterns in macronutrient consumption, dietary restrictions (e.g., vegetarian, gluten-free), food sensitivities, hydration practices, and use of other supplements. The system further collects indicators of inflammation and musculoskeletal health through targeted questions addressing joint pain, frequency of discomfort, and any diagnosed inflammatory conditions. The intake form may also collect information on chronic medical conditions, prior injuries, hormonal health, digestive health, or medication use that may impact nutrient bioavailability, safety of supplementation, or dosing strategies.

In addition to questionnaire-derived inputs, the user profile may include laboratory-derived biological data. This includes, but is not limited to, genotyping results identifying single nucleotide polymorphisms (SNPs) associated with nutrient transport, detoxification efficiency, or metabolic pathways; blood test results such as micronutrient concentrations (e.g., vitamin D, B12, ferritin), inflammatory markers (e.g., CRP), lipid panels (e.g., LDL, HDL, triglycerides), and hormonal panels (e.g., cortisol, testosterone, estrogen); and urinalysis values relevant to hydration status, kidney function, or excretory efficiency. These biological inputs may be uploaded directly by the user or transmitted securely via integrations with certified laboratories or healthcare providers.

In some embodiments, biological data is collected from the user through a combination of clinically validated diagnostic testing, third-party integrations, and structured user uploads. The system may receive laboratory-derived biological data from certified diagnostic providers, including results from blood draws, urinalysis, salivary tests, and buccal swabs. These tests are used to analyze biomarkers relevant to nutritional supplementation, such as serum concentrations of vitamins and minerals (e.g., vitamin D, B12, magnesium, ferritin), lipid profiles (e.g., HDL, LDL, triglycerides), hormonal panels (e.g., cortisol, testosterone, estrogen), inflammation markers (e.g., C-reactive protein), and metabolic indicators (e.g., fasting glucose, insulin resistance indices). In some configurations, the user may provide access to their genetic data by uploading raw genotype files obtained from direct-to-consumer genetic testing services, such as those identifying single nucleotide polymorphisms (SNPs) related to nutrient metabolism, detoxification pathways, or mitochondrial function. The system parses these genetic data files and associates relevant variants with known supplement formulation strategies.

In other embodiments, biological data is collected through manual upload of diagnostic reports or health records by the user, which may include scanned bloodwork summaries, PDFs of clinical evaluations, or screenshots from health portals. The system may extract relevant information from these unstructured formats using OCR (Optical Character Recognition) and structured parsing tools. In still further embodiments, the system integrates with external digital health platforms or electronic health record (EHR) systems to import biomarker data directly from healthcare providers or wearable biosensors. This allows for automated ingestion of longitudinal health data such as heart rate variability, resting heart rate, circadian rhythm trends, glucose levels, or other physiological metrics. In some cases, companion diagnostic kits may be provided for at-home biological testing, where the user performs finger-prick blood tests or urine dipsticks, and the results are either transmitted digitally via a mobile app or sent to a lab and later synchronized with the user profile.

Each biological data point collected is timestamped and optionally tagged with metadata indicating its source and confidence level. In certain embodiments, laboratory-verified data is assigned a higher trust ranking than self-reported metrics and is prioritized during supplement recommendation generation. Together, these mechanisms enable the system to build a dynamic biological profile for the user, forming the foundation for data-driven, personalized nutritional formulations that evolve over time with the user's physiological state.

Once collected, this data is stored in a secure, version-controlled data environment and may be tagged with identifiers that enable some comparison, tracking of temporal changes, and compliance with health data privacy regulations. In some embodiments, each data point is assigned a data quality score or confidence level, especially for self-reported inputs, and the overall profile may include derived scores across categories such as cardiovascular support, recovery, energy metabolism, inflammation, and gut health. The user profile serves as the central input for supplement formulation personalization. It may be continuously updated as new biological results are added, user behavior changes, or new research becomes available. In this way, the user profile is not a static record but a dynamic, evolving digital twin of the user's health and performance state, enabling data-driven refinement of nutritional strategies over time.

At block 120, the processor generates a digital user profile based on the biological data received. The digital user profile is a structured data representation that captures a wide range of individual physiological and metabolic characteristics derived from one or more biological inputs. This profile may be stored within a relational or non-relational database architecture and is formatted to support modular expansion as new data types are added. The processor organizes and maps the collected biomarker values to corresponding physiological performance indicators or biochemical pathways, enabling targeted nutritional intervention. For example, a low serum ferritin level may be mapped to iron metabolism and oxygen transport efficiency, while elevated homocysteine may be associated with methylation pathway imbalances that influence cardiovascular risk and nutrient assimilation. Similarly, a deficiency in vitamin D, combined with genetic polymorphisms in the VDR gene, may indicate a need for adjusted dosage or enhanced bioavailability formulations in the supplement protocol.

The user profile may include indexed fields for demographic data (e.g., age, sex), genotype markers (e.g., SNPs related to COMT, MTHFR, APOE), real-time biomarker levels (e.g., fasting glucose, triglycerides, hs-CRP), hormonal indicators (e.g., testosterone, estradiol), and organ function metrics (e.g., creatinine clearance, liver enzymes). Each of these inputs may be normalized against reference ranges or stratified based on lifestyle factors such as training volume or reported dietary habits. The system may also compute derivative metrics, such as metabolic efficiency scores, oxidative stress indices, or inflammation risk ratings, using a combination of rule-based logic and statistical modeling.

In some embodiments, user classification or clustering algorithms are executed on the generated profile to assign the user to a particular phenotype or archetype. These archetypes may be derived from a pre-trained machine learning model using unsupervised techniques such as k-means clustering, hierarchical agglomeration, or Gaussian mixture models. Classification may segment users into categories such as “high-oxidative stress responder,” “slow methylator,” “high-recovery demand athlete,” or “lipid-sensitive metabolizer,” based on population-level biometric comparison across historical data sets. Such classifications can be used to fine-tune supplement formulations, dosage intervals, or delivery mechanisms. For instance, users clustered into a “fast-caffeine metabolizer” archetype may be assigned a different pre-workout profile than those categorized as “caffeine-sensitive slow metabolizers,” despite similar subjective fatigue levels. The structured digital user profile created at block 120 thus functions not only as a personalized data container but as a computationally actionable object within the supplement optimization pipeline. It enables downstream personalization logic to operate on biologically meaningful patterns and promotes adaptive response to updated test results or ongoing performance feedback. This capability distinguishes the system from generic rule-based supplementation models and enables a truly dynamic, evidence-based approach to nutritional support.

In some embodiments, the system includes functionality for generating and structuring a digital user profile based on biological data collected from the user. This digital user profile comprises a structured data representation that captures a range of individualized physiological and metabolic characteristics derived from laboratory results, genetic tests, and other biometric sources. The system organizes and maps these inputs to corresponding biological functions and metabolic pathways to enable targeted supplementation strategies. For example, biomarkers such as serum vitamin D levels, homocysteine concentrations, lipid profiles, or cortisol levels are linked to categories such as immune support, methylation efficiency, cardiovascular health, and stress response, respectively. The profile may include demographic data such as age and sex, along with genotypic information (e.g., SNP variants related to nutrient metabolism), blood chemistry data, and urinalysis indicators.

The user profile is stored in a format that supports real-time computation and updates, enabling the system to incorporate new biological data as it becomes available. In some embodiments, the system applies clustering or classification algorithms to segment users into phenotype or archetype categories based on biometric trends observed across a broader user population. These categories may include designations such as “high oxidative stress responder,” “slow methylator,” or “lipid-sensitive profile,” each associated with specific nutritional needs. The classification process enhances computational efficiency by narrowing the formulation space and allowing for more precise tailoring of supplements based on biological relevance.

The structured digital profile provides a technical solution to the problem of manual or generalized supplementation by enabling data-driven, automated personalization that is adaptive over time. As new test results or performance feedback are received, the system dynamically updates the user profile and recalculates supplement formulations. This longitudinal tracking allows for early detection of physiological changes and supports continuous optimization of nutritional interventions. Additionally, by structuring the profile in a secure, queryable format, the system enhances traceability, version control, and auditability of user data across the supplementation lifecycle. From a technical perspective, this structured profiling approach improves the functioning of the computerized system by enabling accurate, scalable personalization of supplement protocols based on biological inputs. The system's ability to process and act upon individualized physiological data in an automated fashion represents a practical application of computing resources to improve health-related recommendations and user outcomes.

At block 130, the processor further receives structured qualitative data that complements the biological input collected at earlier stages. This qualitative data encompasses user-entered information that provides critical context for interpreting biological parameters and tailoring supplement recommendations accordingly. Examples of such data include dietary preferences (e.g., vegetarian, vegan, ketogenic, paleo), dietary restrictions (e.g., gluten-free, lactose intolerance, nut allergies), supplement delivery preferences (e.g., capsules, powders, chewables), and specific allergen exclusions. Additionally, users may input subjective health goals such as enhancing endurance, improving sleep quality, reducing fatigue, increasing cognitive performance, enhancing recovery speed, or supporting hormone balance. These self-reported objectives guide the system in prioritizing particular supplement categories, such as adaptogens, nootropics, mitochondrial cofactors, or anti-inflammatory agents, in the formulation logic executed downstream.

Performance feedback may also be collected in real time or over longitudinal intervals, capturing trends in perceived energy levels, muscle soreness, gastrointestinal tolerance, focus during training, or sleep efficiency. This data may be gathered via interactive digital questionnaires, in-app tracking tools, or wearable integrations and subsequently normalized into numerical scales (e.g., 1-10 rating for fatigue) or categorical descriptors (e.g., “high”, “moderate”, “low” recovery status). In some embodiments, users may complete daily or weekly wellness check-ins that are automatically processed and incorporated into the user profile to enable continuous monitoring. For instance, if a user reports a persistent drop in post-exercise recovery despite optimized biomarker values, the system may infer a need for additional support in areas such as cortisol modulation or protein synthesis and adjust the supplement formulation accordingly.

The structured qualitative data enhances the resolution of the personalization model by capturing information that may not be directly observable through biological assays alone. For example, while biomarkers may suggest adequate magnesium status, reported symptoms such as muscle cramps or poor sleep quality may justify the inclusion of bioavailable forms of magnesium (e.g., glycinate or threonate) to support neuromuscular function. Similarly, a user following a ketogenic diet may require adjusted dosages of electrolytes or fat-soluble nutrients due to altered absorption or metabolic utilization. By integrating structured qualitative input alongside clinical biometrics, the system ensures that supplement recommendations are not only biochemically appropriate but also aligned with user-specific lifestyle, goals, and tolerability, thereby delivering a more complete and user-centered personalization framework. This approach enhances the technical capability of the system to adapt in real time to diverse user conditions and preferences, improving long-term adherence and optimizing physiological outcomes.

At block 140, the processor generates a manufacturing instruction set that is tailored to the individual user based on the aggregated user profile, which includes both biological inputs and structured qualitative data. This instruction set provides precise, executable commands to the supplement manufacturing system, enabling the production of a custom supplement formulation designed to address the user's unique physiological profile and personal goals. The manufacturing instruction set includes specifications for active ingredients, their respective dosages, delivery mechanisms, and administration timing. For example, if the user's biological profile reveals a vitamin D deficiency along with low magnesium levels, and the user's dietary inputs indicate they follow a vegan diet, the instruction set may call for plant-derived vitamin D3 (e.g., from lichen) and a highly bioavailable magnesium chelate such as magnesium glycinate, avoiding animal-derived gelatin capsules.

The processor selects and quantifies ingredients from a predefined database of clinically validated compounds, which may include vitamins (e.g., B-complex, D3, K2), minerals (e.g., zinc, selenium, magnesium), amino acids (e.g., L-glutamine, L-theanine), botanicals (e.g., ashwagandha, Rhodiola rosea, turmeric extract), probiotics (e.g., Lactobacillus rhamnosus GG), and specialty compounds (e.g., CoQ10, NAD+ precursors, omega-3 fatty acids, astaxanthin). Each selected component is assigned a dosage range based on the user's biomarker levels, phenotypic classification, symptom reporting, and historical outcome data. For instance, a user reporting frequent fatigue with low serum ferritin and menstruation history may be prescribed an iron bisglycinate formulation with added vitamin C for improved absorption. If gastrointestinal discomfort is reported, the instruction set may recommend enteric-coated delivery or a divided dose schedule to enhance tolerability.

In addition to ingredient selection and dosage, the instruction set also specifies the delivery format, whether the formulation will be compounded as a capsule, powder, liquid tincture, chewable, and/or gel sachet, based on user-entered preferences and dietary restrictions. In some embodiments, an optimal absorption may be optimized, e.g., fat soluble vitamins and supplements such as astaxanthin may be optimally administered for maximal intestinal absorbance.

A user indicating difficulty swallowing capsules and a preference for fast-acting supplements may receive their formulation as a flavored powdered drink mix or liquid solution. The system may also define a timing schedule optimized for circadian rhythms, additives that enhance nutrient absorption, or optimizing training cycles. For example, pre-workout components such as caffeine, beta-alanine, or citrulline may be recommended for morning ingestion, while recovery or sleep-enhancing agents like magnesium, glycine, and GABA may be scheduled for evening use.

The generation of the manufacturing instruction set may be accomplished using a rules-based decision engine, a statistical algorithm trained on aggregated user outcome data, or a machine learning model such as a gradient-boosted tree or neural network that has been trained on historical profiles, formulations, and observed efficacy outcomes. These computational approaches enable precise tuning of formulations based on the nuanced interactions of user data, rather than relying solely on static or manually curated formulas. In some embodiments, the system incorporates formulation logic that accounts for ingredient synergies, contraindications, or competition for absorption, ensuring both safety and effectiveness. For example, the system may avoid combining high levels of calcium and iron in the same dose due to known absorption interference, or it may deliberately pair curcumin with piperine to enhance bioavailability.

By producing a manufacturing instruction set that directly reflects the user's unique biological markers, preferences, and performance feedback, the system delivers highly personalized and clinically relevant supplement solutions. This capability not only enhances user adherence and satisfaction but also represents a technical advancement in computer-enabled supplement design, bridging the gap between laboratory diagnostics, user-reported outcomes, and customized manufacturing processes.

In some embodiments, the system executes one or more algorithms to generate a tailored supplement formulation based on the structured digital user profile created in block 120. The digital user profile comprises a data-rich, structured representation of the user's biological and demographic information, including but not limited to serum biomarker concentrations, genetic markers (e.g., SNPs relevant to nutrient metabolism), metabolic panel results, and reported lifestyle or training metrics. This structured profile enables the system to computationally map biological parameters to relevant physiological domains, such as methylation efficiency, oxidative stress load, hormonal balance, or cardiovascular function, which in turn inform the selection, dosage, and delivery format of nutritional supplement components. In some embodiments, the system applies clustering algorithms or phenotype classification models to identify archetypal user categories, allowing the supplement recommendation engine to operate efficiently across population-level trends while still honoring individual specificity. For example, a user identified as a “high oxidative stress responder” based on elevated CRP and low glutathione levels may be algorithmically matched to a formulation emphasizing antioxidant support. The formulation logic executed at block 150 integrates this stratified data with formulation rules, dosage constraints, ingredient interaction guidelines, and regulatory compliance protocols to produce a biologically coherent and personalized supplement recommendation. This process provides a technical improvement over conventional static or one-size-fits-all approaches, enabling scalable, adaptive, and precision-driven formulation of nutritional protocols based on dynamic user data inputs.

In some embodiments of block 140, the generation of the manufacturing instruction set further incorporates a data-driven customization layer that leverages demographic inputs in combination with biological and qualitative data to refine supplement recommendations at a highly granular level. This capability supports the tailoring of supplement formulations not only to the user's internal biomarkers and health goals, but also to externally observable characteristics and lifestyle factors that influence nutritional demand. The system integrates a range of demographic data points, such as age, biological sex, training level, and athletic discipline, into the supplement formulation logic to ensure relevance, safety, and efficacy across user segments. For instance, age-specific formulation adjustments are implemented to accommodate varying physiological needs over the lifespan. Youth athletes may receive lower dosages and avoid certain botanicals, whereas older athletes (e.g., masters-level competitors) may be prescribed formulations that emphasize joint support, cardiovascular health, or hormonal balance. Similarly, sex-specific recommendations are made based on known differences in nutritional requirements. Female users with low ferritin levels and regular menstruation may receive iron supplementation with added vitamin C to support absorption, while male users reporting signs of testosterone deficiency may receive zinc, boron, or adaptogenic support such as ashwagandha.

The system also classifies users by their athletic engagement level, such as recreational, intermediate, advanced, or elite, based on structured questionnaire responses and historical performance inputs. This classification informs intensity-specific formulations, where elite athletes may receive compounds supporting mitochondrial efficiency and recovery acceleration (e.g., CoQ10, NAD+ precursors), while recreational users may receive foundational wellness support. Further, the type of training performed (e.g., endurance, resistance training, high-intensity interval training (HIIT), or team sports) guides the selection of conditionally essential nutrients. For example, endurance athletes may receive higher doses of electrolytes and oxidative stress modulators such as astaxanthin, which has been shown to support mitochondrial function and reduce exercise-induced oxidative damage. In contrast, resistance-trained individuals may benefit more from targeted support including creatine, beta-alanine, and branched-chain amino acids. The processor automatically incorporates these demographic and activity-based considerations when generating the manufacturing instruction set, which includes selected ingredients, dosage levels, delivery format, and timing instructions. This expanded capability enhances the system's precision by accounting for both internal (biological) and external (demographic and behavioral) data streams. The resulting formulation reflects not only the user's biomarker trends and health goals, but also real-world activity demands and demographic profiles. This integration of contextual intelligence into the formulation engine marks a distinct technical improvement in personalized health systems, enabling a higher-resolution model of supplement customization that goes beyond static user data to dynamically address complex, multifactorial user needs.

As part of block 140, the system employs proprietary algorithms to generate the manufacturing instruction set by translating the user's structured biological, qualitative, and demographic data into a personalized supplement formulation. These algorithms form a key element of the customization engine and represent a technical innovation that distinguishes the system from conventional rule-based health advisory platforms. At the core of the system is a multi-stage scoring algorithm that quantifies the relevance of each supplement ingredient across various performance and health-related categories, such as energy metabolism, recovery optimization, oxidative stress and inflammation control, cognitive function, and hormonal support.

The process begins with normalized response weighting, where each data input from the user profile, whether a biomarker value, training frequency, reported symptom, or dietary preference, is transformed onto a standardized scale, typically ranging from 0 to 1. This normalization allows disparate data types (e.g., cortisol levels, subjective fatigue scores, magnesium intake) to be evaluated on a unified scale, facilitating quantitative comparisons and algorithmic processing. Each response is then subjected to category impact multiplication, which applies a set of predefined or learned weights that reflect the relative influence of a particular variable on specific physiological domains. For example, a high score in self-reported stress and elevated cortisol levels may heavily weight the “adrenal support” and “sleep optimization” categories, thus influencing downstream supplement recommendations such as phosphatidylserine or ashwagandha.

The algorithm further executes conditional trigger evaluations to identify when thresholds or critical data patterns are met, triggering the inclusion or exclusion of specific ingredients. For instance, the presence of elevated homocysteine combined with a methylation SNP (such as MTHFR C677T) may activate a trigger to include methylated forms of B12 and folate. Similarly, if a user has a known intolerance to histamines and reports gastrointestinal discomfort, the system may suppress the inclusion of certain probiotics or fermented adaptogens. Athlete-specific boost calculations are then applied to fine-tune dosages and ingredient pairings based on the user's declared athletic classification, such as increasing creatine and taurine dosage for high-intensity resistance athletes or adding astaxanthin, omega-3s and curcumin for endurance athletes with joint stress.

Finally, the algorithm computes a final composite score for each potential supplement using a ranking function that integrates weighted inputs across all applicable categories. This final score determines the selection and prioritization of compounds for inclusion in the supplement formulation, which is output as part of the manufacturing instruction set. The algorithm ensures that each ingredient is biologically relevant, contextually justified, and quantitatively optimized for the individual user.

By employing this algorithmic pipeline, the system delivers high-resolution, adaptive supplement recommendations that are not possible through static decision trees or generic expert systems. The proprietary logic enables integration of complex multidimensional data into a coherent output that enhances personalization, efficacy, and safety. This use of advanced computational methods to interpret biological and qualitative inputs and to automate the formulation of personalized supplements constitutes a technical advancement in computer-enabled health systems.

At block 150, the manufacturing instruction set generated from the individualized user profile is transmitted to a supplement compounding system for execution. This transmission may occur via a secure, encrypted data pipeline that ensures integrity and confidentiality of the instruction set as it travels between the central processing system and the physical production site. The compounding system may be located on-site within a vertically integrated production facility or at a remote contract manufacturer operating under Good Manufacturing Practices (GMP) and connected to the system via application programming interfaces (APIs) or proprietary middleware. The compounding system includes pharmaceutical-grade automated machinery that executes multiple stages of the supplement preparation workflow, including the weighing, blending, dosing, encapsulating, sealing, and labeling of the final supplement product.

Each manufactured batch is uniquely tagged with a machine-readable batch identifier, production date, and version number corresponding to the specific manufacturing instruction set and user profile from which it originated. This traceability feature supports downstream auditing, inventory control, and safety recall procedures, and ensures compliance with regulatory frameworks such as FDA 21 CFR Part 111 for dietary supplements. In some embodiments, radio-frequency identification (RFID) tags or QR codes may be printed on the product packaging to facilitate digital traceability and user-facing interaction, such as product authentication or reordering.

The compounding and delivery process is designed to be iterative, allowing the formulation to evolve over time as additional biomarker data, lifestyle inputs, and feedback are collected from the user. For instance, a user whose follow-up bloodwork reveals normalized vitamin B12 levels may receive a revised formulation with a maintenance-level dose, while a user reporting continued fatigue may be recommended a new blend including NAD+ precursors, adaptogenic herbs, and recovery-supporting compounds such as astaxanthin to help mitigate oxidative stress and promote cellular resilience. The system's dynamic formulation capability enables longitudinal personalization, aligning with seasonal training cycles, health objectives, or recovery status.

In further embodiments, the system incorporates a compliance monitoring module that tracks user adherence to the prescribed supplementation protocol. This module may integrate data from smart packaging, app-based check-ins, or wearables, and can alert users or healthcare professionals in the event of missed doses or inconsistent usage patterns. Additionally, a digital credentialing mechanism, such as a “digital pass,” is provided to authenticated users. This digital pass grants secure access via mobile device to key formulation information, including active ingredient lists, dosage schedules, timing instructions (e.g., morning, pre-workout, bedtime), and potential interactions or contraindications. The digital pass may also be updated in real time to reflect revised protocols based on new data, ensuring that users always have current, personalized supplement instructions at their fingertips.

By tightly integrating the manufacturing system with personalized data inputs, version-controlled protocols, and user-facing digital tools, block 150 achieves a level of end-to-end personalization and traceability that significantly exceeds the capabilities of traditional supplement manufacturing. This results in greater transparency, improved safety, and more effective health outcomes, representing a meaningful technical improvement in the field of digital health and personalized nutrition.

By automating the collection, interpretation, and application of complex biological and behavioral data, the system described in FIG. 1 enables dynamic, data-driven, and individualized supplement recommendations that go beyond static, one-size-fits-all approaches. The integration of structured input, algorithmic processing, and physical product generation provides a scalable, clinically-informed platform for precision supplementation.

Some embodiments of the method illustrated in FIG. 1 further include the use of a machine learning algorithm to assist in the generation of the personalized supplement formulation. In such embodiments, after receiving the biological data and generating the digital user profile, the system may invoke a machine learning engine that has been trained on a corpus of prior genetic, biochemical, and outcome data. This engine is configured to identify correlations between specific genetic polymorphisms, such as single nucleotide polymorphisms (SNPs), and nutrient metabolism patterns, supplement tolerability, or absorption efficiency. The model may also use dimensionality reduction, supervised classification, or clustering techniques to segment users into supplement response categories based on biochemical phenotype.

In some configurations, the machine learning system operates as an automated diagnostic module that registers genetic markers and incorporates demographic variables such as age, gender, ethnicity, and activity level. These demographic attributes are used in conjunction with the user's biomarker profile to select, from a structured database of prior cases and ingredient combinations, one or more optimal treatment paths for nutritional supplementation. For example, the model may match a user with a variant in the MTHFR gene to a methylated folate formulation, or identify users with impaired lipid transport efficiency for tailored omega-3 dosing protocols. This automated diagnostic process reduces reliance on manual interpretation and increases consistency and personalization across large-scale user populations.

The system may also incorporate an iterative development framework wherein supplement formulations are refined over time based on newly available research, updated user data, and ongoing user-reported feedback. This adaptive cycle allows for reprocessing the user profile at regular intervals, such as upon receipt of updated lab data or structured feedback indicating suboptimal response, and for automatically modifying ingredient selection or dosage in response to evolving needs. For example, if a user's inflammatory markers improve or training demands increase, the formulation may be updated to reflect recovery needs or increased micronutrient demand including, but not limited to, astaxanthin (preferably at least about 12 mg-24 mg).

Biological analysis used in this method may combine genetic testing with additional physiological assessments, including blood metabolomics, lipidomics, urinalysis, and in some embodiments, sweat composition or salivary biomarker tracking. The outputs of these tests may be used to identify nutrient deficiencies, metabolic imbalances, or absorption inefficiencies. Supplement formulations generated by the system are intended to be bioavailable and are designed to match the user's specific absorption pathways and biochemical needs. In some implementations, ingredient bioavailability is matched to transporter gene variants or cellular uptake efficiency.

The data-driven customization of supplement regimens extends beyond purely biological analysis and includes the integration of demographic and behavioral information, which may affect supplement timing, delivery format, or tolerability. For instance, a high-performance athlete undergoing high-frequency training cycles may receive a formulation with adaptogens and mitochondrial support compounds, including, but not limited to, astaxanthin whereas a sedentary individual with metabolic syndrome may be recommended formulations supporting insulin sensitivity and anti-inflammatory balance including, but not limited to, astaxanthin. The formulation engine may leverage a proprietary algorithmic repository, a set of machine learning models trained on longitudinal case histories and validated with user-reported outcome data. This repository may comprise rule-based sub models as well as continuously trained models that ingest anonymized user feedback to refine recommendation precision over time.

In some embodiments, the system implements an integrated biological analysis (including metabolomic, lipidomic, urinary, and genetic datasets) to generate individual supplement protocols; the proprietary diagnostic and recommendation algorithms that translate this data into tailored ingredient profiles; and the specific, non-generic combinations of nutrients selected and adjusted based on biological need, genetic compatibility, and phenotypic variability. These combinations may include dosing strategies, ingredient synergies, or exclusion filters that are not present in conventional or population-level supplement formulations. FIG. 1 not only describes a static method of generating a supplement formulation based on user profile data but also encompasses embodiments where adaptive, intelligent computation systems play a central role in automating, optimizing, and personalizing supplement generation and delivery in real time across diverse user populations.

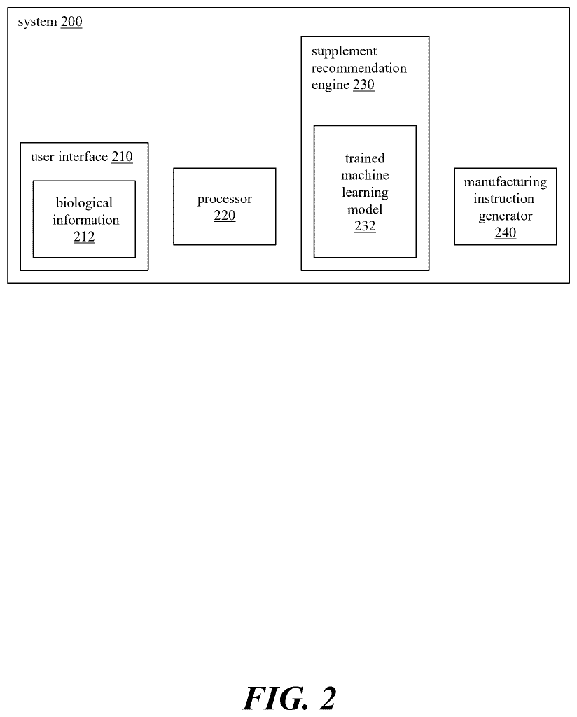

FIG. 2 illustrates an example embodiment of a system 200 for generating personalized nutritional supplement formulations using user-specific biological and behavioral input data. The system 200 is configured to integrate, process, and apply heterogeneous data sources through a combination of user input modules, algorithmic recommendation engines, and manufacturing logic.

The system 200 includes a user interface 210, which may comprise a web-based platform, mobile application, tablet display, or kiosk interface configured to receive user-supplied information. In one embodiment, the user interface 210 includes a biological information module 212, which is configured to ingest biological data such as genetic profiles (e.g., single nucleotide polymorphisms), blood biomarker readings, lipid panels, or urine metabolite results. This biological data may be input manually or retrieved from third-party laboratory databases through secure API integration.

A processor 220 is operatively coupled to the user interface 210 and serves as the central computing unit responsible for executing instructions associated with supplement analysis and recommendation. The processor 220 may be a central processing unit (CPU), a graphical processing unit (GPU), or a neural processing unit (NPU) capable of performing high-throughput analysis and machine learning inference tasks.

The processor 220 communicates with a supplement recommendation engine 230. The supplement recommendation engine 230 is configured to analyze the user profile and generate one or more candidate supplement formulations. In the illustrated embodiment, the supplement recommendation engine 230 includes a trained machine learning model 232. The trained machine learning model 232 may be a supervised learning model, such as a convolutional neural network (CNN), decision tree, support vector machine, or ensemble learning architecture trained on historical datasets of user biometrics, supplement compositions, and associated outcome metrics. Alternatively, the model 232 may be implemented as a reinforcement learning agent that optimizes formulation recommendations through ongoing feedback and results monitoring.

The machine learning model 232 is configured to process both the biological inputs from module 212 and structured qualitative feedback such as user-reported energy levels, training goals, or dietary constraints. These multidimensional inputs are normalized and analyzed by the engine 230 to produce an individualized formulation profile. Based on the output of the recommendation engine 230, the system 200 further comprises a manufacturing instruction generator 240. The manufacturing instruction generator 240 translates the recommended supplement formulation into a structured instruction set compatible with a compounding or formulation system. This instruction set may define a list of active ingredients, micro- or macronutrient quantities, delivery medium (e.g., capsule, powder, liquid), and packaging configuration. The instruction set may also specify any allergen filters or formulation exclusions based on user sensitivities.

In various embodiments, the manufacturing instruction generator 240 outputs machine-readable instructions, such as JSON, XML, or G-code, and transmits such data to an automated or semi-automated compounding device. The generator 240 may further include a compliance module that assigns batch IDs, timestamps, and protocol metadata for traceability and auditability.

The system 200 may operate in a cloud environment, on a distributed network, or on a local computing device depending on deployment preferences. It may include additional modules not shown, such as user compliance tracking systems, integration with wearable health devices, and periodic update mechanisms for refining supplement profiles based on evolving biomarker data. Altogether, the system 200 presents a modular, scalable architecture for delivering biologically personalized nutrition recommendations that are tightly integrated with production instructions, thereby enabling an end-to-end adaptive supplementation platform.

In some embodiments, the system includes a framework designed to support some integration of advanced biological data sources, including blood metabolic testing, lipid analysis, and urinalysis. While current embodiments may rely primarily on user-reported data and preconfigured logic rules, the system is architected to accept and process clinical biomarker data to enhance the precision and personalization of supplement recommendations. In these embodiments, biological input fields such as inflammatory markers, hormone levels, and nutrient serum concentrations (e.g., vitamin D, ferritin, B12) may be received from certified diagnostic laboratories and stored in a structured format within the user profile. The system may apply interpretative thresholds and ranges that reflect both general population health norms and sport-specific metabolic performance requirements. For example, in certain embodiments, the system may interpret hemoglobin or VO₂-max related parameters in conjunction with endurance athlete classifications to tailor iron supplementation levels.

In some embodiments, medical condition constraints are implemented as exclusionary logic gates within the recommendation engine to prevent the inclusion of contraindicated compounds. For instance, a user profile indicating a history of autoimmune disease may trigger suppression of ingredients with immunostimulatory properties, such as astragalus or high-dose echinacea. These constraints may be dynamically applied by a rules-based engine or a machine-learning model trained to identify high-risk ingredient-user interactions. In further embodiments, protein intake requirements may be calculated based on user-specific variables, including body weight, training modality, and recovery status, although dynamic protein advisory recommendations may not yet be operationalized in current implementations.

With regard to iterative development, some embodiments include a dynamic feedback and adaptation module configured to enable continuous refinement of supplement formulations. Although initial embodiments may provide static recommendations, the system is configured to implement longitudinal monitoring through user check-ins, performance reporting tools, and, in some embodiments, biomarker trend analysis. In certain implementations, the system may prompt users to complete digital assessments within a defined interval prior to the dispatch of a subsequent supplement package. These assessments may capture data regarding symptom resolution, energy levels, gastrointestinal tolerance, or perceived performance. The resulting data may be used by the personalization engine to recalibrate ingredient selection, dosage strength, or delivery timing for the next formulation cycle.

In some embodiments, a visual performance dashboard is provided to track user outcomes across time, with integrated data visualization tools allowing both the user and the system to assess progress toward predefined health and performance goals. This dashboard may incorporate adherence tracking, self-reported effects, and eventually quantitative metrics from integrated wearables or diagnostic labs. By incorporating these mechanisms, the system is structured to support closed-loop, adaptive nutrition recommendations that evolve in response to verified outcomes, thereby improving precision, engagement, and physiological alignment of the supplementation protocol.

In some embodiments, the system is designed to incorporate machine learning capabilities to enhance the tailoring of nutritional supplement formulations based on user-specific biological and qualitative data inputs. Although initial implementations rely on rule-based logic and static scoring algorithms, some embodiments are configured to support model training using anonymized user response data. In such embodiments, a machine learning framework—potentially integrated through a cloud-based service such as Google Cloud Model Garden—receives user inputs and associated outcome labels to optimize supplement efficacy predictions and to generate explanatory outputs for end-user education. These embodiments enable the system to identify meaningful patterns in response data using k-means clustering or weighted clustering techniques across population subsets. For example, the system may identify that users with a specific biomarker pattern and training profile respond more favorably to certain adaptogenic compounds. This predictive insight is used to adjust supplement recommendations over time, thereby improving the accuracy and efficacy of personalization.

Additional embodiments include the use of machine learning to automate the recognition and interpretation of genetic single-nucleotide polymorphisms (SNPs). Although current implementations do not process genetic data, the system architecture is prepared to ingest genetic test results and evaluate the relevance of key SNPs, such as those related to MTHFR, COMT, or CYP450 pathways, for supplement selection. In some embodiments, SNP profiles are mapped to supplement algorithms that determine ingredient suitability and dosing modifications based on known genetic predispositions, with potential pharmacogenomic applications in both nutrient metabolism and sensitivity prediction. Genetic datasets collected in early trials may serve as the training foundation for some model deployment.

In further embodiments, the system is configured to perform advanced biological analysis integrating multiple biomarker classes, including blood metabolomic data, lipidomic profiles, and urinalysis metrics. Though not active in initial system versions, laboratory data integration APIs are planned to allow automatic ingestion of structured test results from third-party vendors. In these embodiments, biomarkers such as hs-CRP, oxLDL, ApoB, and LDL/HDL ratios, homocysteine levels, liver enzyme activity, or urinary electrolyte balance are analyzed in relation to wellness thresholds and interpreted within a multidimensional scoring matrix. This data may be processed by proprietary algorithms developed by a multidisciplinary team of metabolic biologists, dietitians, and physiologists, who have contributed interpretive frameworks that power real-time recommendation adjustments. Moreover, some embodiments may integrate multi-omics processing across qualitative and quantitative data streams, allowing the decision engine to synthesize context from lipid, blood, and lifestyle indicators to inform supplement formulation logic. This capability will enable the system to dynamically cross-reference biological, genetic, and experiential signals to drive adaptive personalization of formulations, thereby offering an advancement in the technical field of precision nutrition and data-driven health optimization.

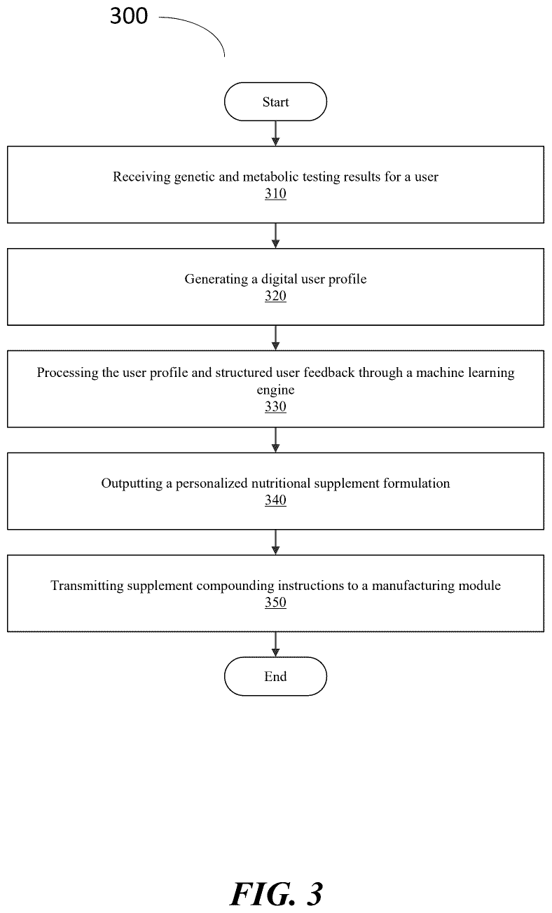

FIG. 3 illustrates a method 300 for generating and delivering a personalized nutritional supplement formulation using biological testing results, user-specific feedback, and machine learning-driven optimization. The method is implemented by one or more computing systems configured to acquire, process, and integrate diverse data inputs to produce a highly individualized supplementation protocol aligned with the user's physiological profile and health objectives.

The process initiates at step 310, where the system receives genetic and metabolic testing results corresponding to a specific user. In some embodiments, the genetic data includes single nucleotide polymorphism (SNP) profiles relevant to nutrient absorption, enzymatic activity, or metabolic regulation—such as polymorphisms in the MTHFR, APOE, or COMT genes. Concurrently, metabolic testing results may include serum and plasma concentrations of vitamins, minerals, and metabolites such as vitamin D, iron, B12, homocysteine, fasting glucose, hs-CRP, and cortisol, along with lipid panel values including ox-LDL, ApoB, LDL, HDL, triglycerides, and total cholesterol. Additional biomarker data may be captured from hormone panels (e.g., testosterone, estrogen, thyroid markers) and urinalysis (e.g., urinary creatinine, ketones, specific gravity).

These biological inputs may be received via secure data connections with third-party laboratory information management systems (LIMS), electronic health record (EHR) interfaces compliant with HL7 or FHIR protocols, or user-initiated uploads of PDF or structured data files containing lab results. In some embodiments, the system supports guided lab ordering workflows, enabling users to initiate testing through partner diagnostics providers, with results automatically mapped to the corresponding user profile upon completion.

The biomarkers received in step 310 serve as the objective, data-driven foundation upon which the nutritional analysis and recommendation engine operates. The collected values are interpreted in relation to medically accepted reference ranges as well as performance-optimized thresholds when athletic profiles are indicated. For example, a user with suboptimal omega-3 index, elevated inflammatory markers, and high cortisol may be identified as a candidate for formulations targeting inflammation modulation, adrenal support, and cognitive resilience. In such embodiments, the system may further contextualize the raw data by correlating trends across multiple biomarker classes, flagging potential nutrient deficiencies, and activating supplement formulation rules specific to the user's physiological and lifestyle profile.

At step 320, a digital user profile is generated by the system based on the biological data collected in step 310. This digital profile is a structured, machine-readable dataset that encapsulates the user's unique physiological and metabolic characteristics. The system organizes the received inputs into predefined data schemas, mapping each biomarker to corresponding physiological systems or supplement-relevant domains. For instance, vitamin D levels may be categorized under immune and skeletal health, while ferritin and hemoglobin levels inform oxygen transport and recovery capacity. In some embodiments, the system performs automated calculations to generate derived health indices. These may include nutrient ratio scores (e.g., calcium-to-magnesium or omega-6 to omega-3 ratios), metabolic efficiency scores, or estimations of biological or metabolic age based on patterns in hormone levels, inflammation markers, and lipid metabolism. These derived metrics are algorithmically benchmarked against both clinical thresholds and normative datasets drawn from athletic or general population cohorts.

The profile also tags biologically relevant features that inform supplement customization. For example, low serum B12 combined with a homozygous MTHFR mutation may trigger the recommendation of a methylated B-complex rather than a standard form. Similarly, signs of adrenal stress, such as elevated cortisol and low DHEA, may prompt adaptogen inclusion. The system assigns metadata to each data point, including the source laboratory, method of analysis, collection date, and validation status. This metadata supports traceability and auditability, allowing clinical staff or AI systems to evaluate the reliability and currency of each input.

In some embodiments, the digital profile further incorporates a longitudinal component, enabling the system to track changes over time in key biomarker domains. For example, a user's lipid panel data over several months may show trends of improvement or decline, allowing the system to assess supplement effectiveness and adjust dosages or ingredient compositions in response. All profile data is stored in a secure, version-controlled environment using encryption and access controls compliant with healthcare privacy regulations (e.g., HIPAA, GDPR). This longitudinal, adaptive profile serves as the foundational reference model from which subsequent formulation, personalization, and performance feedback loops are derived.

At step 330, the system processes the digital user profile, comprising biological data and derived health indices, in combination with structured user feedback using a machine learning (ML) engine to generate a personalized supplementation strategy. The structured user feedback consists of self-reported qualitative data gathered through guided digital questionnaires, mobile app interfaces, or web-based assessments. This feedback includes dietary preferences (e.g., vegan, paleo, ketogenic), allergen exclusions (e.g., gluten, soy, dairy), subjective wellness scores (e.g., fatigue levels, digestive discomfort, perceived recovery), sleep quality metrics, stress levels, and user-defined performance goals (e.g., improve endurance, reduce inflammation, enhance cognitive function).

These inputs are converted into structured formats such as numerical scales (e.g., 1-10 sleep rating), categorical variables (e.g., “yes” or “no” to supplement adherence), and Boolean flags (e.g., exclusion of caffeine-based compounds). Natural language inputs may be preprocessed using text classification algorithms or converted via structured intake forms with predefined options, ensuring consistency and compatibility with the ML engine. The machine learning engine itself may take various forms depending on implementation. In some embodiments, it comprises a supervised neural network trained on labeled datasets of prior user profiles and corresponding supplement outcomes. In others, a gradient-boosted ensemble model or hybrid system integrating both decision-tree logic and rule-based nutritional constraints may be used. These models are trained on historical user data, expert-reviewed supplement recommendations, and clinical outcomes where available. The ML engine learns associations between complex input combinations, such as low magnesium, poor sleep quality, and high stress, and effective formulation strategies, such as magnesium glycinate combined with adaptogens like ashwagandha.

The engine may also integrate population-level clustering algorithms, such as k-means or hierarchical agglomerative clustering, to position the user within phenotypic subgroups. For example, an athlete with high cortisol, moderate inflammation, and frequent fatigue may be assigned to a “burnout risk” cluster, triggering tailored recommendations for adrenal and mitochondrial support. The predictive output of the ML engine includes not only the formulation content (e.g., specific dosages and ingredient types) but also contextual confidence scores and expected outcome trajectories (e.g., improved recovery time within four weeks). This technical capability enables dynamic personalization that evolves based on longitudinal data and observed outcomes, creating a feedback-informed supplement system that moves beyond static one-size-fits-all recommendations.

The technical benefit of the method described in step 330 is the transformation of static supplement protocols into intelligent, adaptive nutritional recommendations through the use of machine learning and structured data integration. By processing a combination of biological data, such as genetic markers, blood lipid levels, and hormone concentrations, and structured user feedback, such as wellness scores, dietary preferences, and performance goals, the system creates a dynamic formulation engine capable of continuously optimizing supplement protocols. This computational approach enables the identification of complex, multi-dimensional patterns that would be difficult or impossible to detect through manual analysis or rule-based systems alone. In some embodiments, the use of predictive modeling trained on historical outcome data further enhances personalization accuracy, allowing the system to generate supplement formulations that are specifically tailored to a user's evolving physiological and behavioral profile.

This method offers additional technical advantages including improved scalability, consistency, and traceability of recommendations. The machine learning engine applies consistent logic across large user populations and may generate confidence scores and audit trails for each recommendation, thereby enhancing system transparency and supporting regulatory compliance. Furthermore, the ability to iteratively update recommendations based on longitudinal user data and observed outcomes ensures that the system remains responsive and clinically relevant over time. These features result in a technically superior, computer-implemented system that enhances both the reliability and effectiveness of personalized supplement delivery, representing a practical application of data science that improves upon conventional nutritional advisory methods.

In some embodiments, the machine learning (ML) model processing described herein addresses a technical problem in the field of personalized supplement manufacturing by enabling a data-driven, automated method for transforming heterogeneous biological and qualitative inputs into individualized formulation protocols. The ML engine receives multi-dimensional user data, including genetic variants (e.g., single nucleotide polymorphisms), biomarker values (e.g., serum ferritin, vitamin D levels), demographic attributes (e.g., age, sex, sport type), and structured feedback (e.g., dietary restrictions, symptom reports, performance goals), and processes this data using a trained model architecture. The model may include neural networks, ensemble learners, or hybrid rule-based systems that have been trained on validated datasets correlating supplementation outcomes to specific input profiles. Unlike conventional scoring systems or static recommendation engines, the ML model executes a sequence of technical operations to derive a machine-readable supplement protocol that includes ingredient selection, precise dosage calculations, delivery format (e.g., capsule, powder, or liquid), and administration schedule based on physiological patterns such as circadian rhythm or training phase.

This approach confers several technical advantages. First, it automates the translation of raw health data into precise manufacturing instructions, which traditionally would require expert interpretation. Second, it reduces human error and improves scalability by continuously updating the formulation logic through outcome-based learning. Third, the ML model supports contraindication checks, such as excluding immune stimulants for users with autoimmune predispositions, by identifying and tagging risk factors during inference. Fourth, by employing clustering and pattern recognition, the model enables phenotype-aware supplementation, allowing for nuanced personalization beyond one-size-fits-all regimens. The resulting manufacturing instruction set may be transmitted directly to a compounding system in a structured format, enabling real-time production of individualized supplement packets. Collectively, these elements demonstrate a technical improvement to both the personalization and production of nutritional supplementation, advancing the field beyond abstract classification or data analysis.

In step 340, the system generates and outputs a personalized nutritional supplement formulation tailored to the user's unique biological profile and lifestyle inputs. This output includes a detailed list of recommended ingredients, such as vitamins, minerals, amino acids, adaptogens, probiotics, or botanicals, along with precise dosage amounts calculated based on the user's biomarker levels, genetic variations, and reported wellness metrics. The formulation further specifies the most appropriate delivery format, such as encapsulated pills, powdered blends, liquid tinctures, or soft gels, selected according to factors like gastrointestinal sensitivity, user preference, or bioavailability requirements.

Additionally, the system determines an optimal intake schedule for each formulation component, which may align with the user's daily training phases (e.g., pre-workout, post-workout), circadian rhythm (e.g., morning cortisol peaks, evening melatonin cycles), or reported lifestyle patterns (e.g., shift work, intermittent fasting). In some embodiments, the system includes contextual annotations that explain the rationale behind certain ingredient choices or formulation changes. For example, if the user's genetic data reveals single nucleotide polymorphisms (SNPs) associated with impaired methylation (e.g., MTHFR variants), the system may increase the dosage of methylated B-vitamins such as methylcobalamin (B12) and methylfolate, while simultaneously flagging this adjustment for educational visibility within the user interface. This level of granularity in formulation design not only ensures biological coherence and personal relevance but also enhances user adherence and engagement by transparently linking recommendations to personal health data. The result is a highly customized, evidence-based supplement protocol that evolves in tandem with the user's physiological status and performance objectives.

The technical benefit of step 340 lies in the system's ability to generate a highly customized, biologically-coherent supplement formulation through automated data-driven computation, thereby advancing the field of personalized nutrition beyond static or generalized approaches. By integrating diverse data types, including genetic markers, blood metabolite concentrations, user-reported wellness metrics, and lifestyle patterns, the system delivers individualized formulations with precise dosages, delivery formats, and intake schedules. This eliminates the need for manual interpretation or generic supplementation, reducing the risk of ineffective or contraindicated ingredient use.

From a technical standpoint, the system enhances operational efficiency and reliability by automating complex formulation logic using structured rules, algorithmic weighting, and model-driven prediction. It ensures repeatable, scalable precision in tailoring ingredient recommendations to user-specific needs, while simultaneously incorporating feedback loops to improve over time. The inclusion of context-specific logic, such as aligning ingredient delivery to circadian rhythms or training cycles, demonstrates a computationally implemented advancement that is not abstract but grounded in real-world physiological optimization. Furthermore, by providing transparent, explainable output tied directly to biological data (e.g., increased methylated B12 for users with MTHFR variants), the system enhances trust, regulatory defensibility, and user comprehension.

In step 350, the system transmits a finalized set of compounding instructions to a manufacturing module responsible for producing the personalized supplement formulation. These compounding instructions include machine-readable specifications detailing the precise identity, quantity, and concentration of each active and inactive ingredient, as well as formulation parameters such as excipient selection, particle size requirements, solubility constraints, and ingredient interaction tolerances. The output file may conform to standardized data interchange formats (e.g., XML, JSON, or HL7 FHIR extensions) to ensure seamless compatibility with automated pharmaceutical or nutraceutical compounding systems, such as capsule-filling machines, powder-blending equipment, or liquid-dosing apparatus.

In some embodiments, the manufacturing instructions also include individualized labeling data generated from the user profile. This data may specify allergen disclosures (e.g., “contains soy lecithin”), dietary certifications (e.g., vegan, gluten-free, NSF-certified for sport), supplement facts (e.g., daily value percentages), and customized usage protocols (e.g., “take 2 capsules 30 minutes before endurance training”). The system may further append a unique formulation identifier, user ID, batch number, and date of manufacture for traceability and quality control. To ensure regulatory compliance and consumer transparency, additional metadata may include expiration dates based on ingredient stability, cautionary use statements related to medical history (e.g., “not for use in individuals with autoimmune disorders”), and visual identifiers such as QR codes linking to digital product information or tracking portals. This integration of data-driven personalization with manufacturing-level automation enables just-in-time production of highly individualized formulations at scale, reducing waste, improving compliance, and enhancing the therapeutic alignment between formulation and user need.

The technical benefit of step 350 lies in the seamless integration of user-specific computational outputs with automated manufacturing systems to produce individualized nutritional supplement formulations. This step transforms complex, multi-input personalization data into standardized, machine-executable compounding instructions that eliminate manual interpretation and human error, thereby improving accuracy, consistency, and production efficiency in the formulation of health products. By transmitting ingredient specifications, dosage levels, delivery formats, and labeling data in a structured, machine-readable format, the system enables real-time interoperability with pharmaceutical-grade production equipment. This integration allows for precise formulation adjustments based on individual biological and qualitative parameters, such as increasing magnesium dosage for athletes reporting muscle cramping or excluding allergens based on user-reported sensitivities, without the need to redesign the underlying production architecture.

Furthermore, the automated generation of labeling and traceability metadata (e.g., batch IDs, personalized usage notes, and regulatory disclosures) enhances compliance with FDA, GMP, and consumer safety standards. It also supports version control and auditability, enabling long-term monitoring of formulation changes in response to evolving user profiles. The method illustrated in FIG. 3 supports a scalable and adaptive personalization system for nutritional supplements. It combines laboratory-grade biomarker analysis, user-centered data collection, and machine learning to deliver real-time, individualized formulations. This end-to-end process ensures that recommendations are rooted in biological evidence and dynamically updated to reflect changes in physiology or user input over time.

User data may be sourced not only from laboratory tests and self-reported inputs but also from wearable devices that continuously monitor physiological metrics. These devices can capture real-time data such as heart rate variability, sleep quality, activity levels, skin temperature, and stress indicators. When integrated into the personalization engine, this streaming data provides a dynamic, longitudinal view of the user's health status, enabling the system to detect trends, deviations, and recovery patterns that may not be evident from periodic lab testing alone. This continuous feedback loop allows for more timely and responsive adjustments to supplement formulations, supporting a truly adaptive and proactive approach to wellness management.

### EXAMPLES

In some embodiments, the personalized supplementation system was validated through a pilot feasibility study designed to assess its capacity to enhance athletic performance, recovery, and biological health indicators in endurance athletes. This example involved the deployment of the claimed methods and systems in a real-world athletic cohort in collaboration with the University of California, Berkeley Triathlon Team.

A total of thirty (30) collegiate athletes aged 18-26 years were enrolled, with twenty-two (22) completing the full protocol. All participants were members of the Cal Triathlon Team, encompassing both elite and amateur competitors. The intervention included comprehensive baseline and post-intervention assessments, incorporating biological testing, physiological performance analysis, and personalized supplement administration.

Pre- and post-intervention data collection included a structured Athlete Intake Form that captured user-specific structured qualitative data such as training volume, dietary practices, symptomatology (e.g., delayed onset muscle soreness, GI distress), sleep quality, stress ratings, and perceived wellness metrics. Biological data collection involved fingerstick capillary blood sampling, analyzed for serum levels of Vitamin D, Ferritin, Vitamin B12, hs-CRP, Omega-3 index, Magnesium, and additional biomarkers relevant to athletic recovery and performance. Genetic data were also collected via whole genome sequencing for retrospective correlation with supplementation outcomes.

Each participant underwent exercise physiology testing at both time points, including VO₂ max assessments and respiratory efficiency testing, which served as objective performance metrics. These physiological outputs were integrated into the participant's digital profile to refine supplement personalization.

The intervention consisted of a custom supplement formulation generated for each athlete using the system's proprietary algorithm, which combined biomarker data with structured user-reported information to derive ingredient selection, dosage amounts, and intake scheduling. Formulations typically included combinations of ergogenic and adaptogenic compounds such as magnesium, vitamin D, astaxanthin, ashwagandha, omega-3 fatty acids, curcumin, and branched-chain amino acids, configured to address deficiencies, modulate inflammation, and support neuromuscular recovery.

Manufacturing was conducted in a cGMP-certified facility, with supplements packaged into individualized daily sachets. Each package included customized labeling indicating formulation composition, usage instructions, and relevant health disclaimers. Users were granted mobile access to their formulation details via a digital pass, enabling real-time review and compliance tracking.

Post-intervention analysis revealed statistically significant improvements across several key performance indicators. Notably, participants demonstrated mean increases in Functional Threshold Power (FTP), VO₂ max, and ventilatory thresholds. Biologically, serum levels of Vitamin D and B12 increased significantly across the cohort. Furthermore, a significant decrease in pro-inflammatory cytokine TNF-a was observed post-intervention, indicating a systemic reduction in inflammation.

Individual-level improvements were also documented. For example, participant OG0027 exhibited a 15.38% increase in FTP, a 7 mL/kg/min gain in VO₂ max, and a doubling of serum ferritin levels, alongside a 50% reduction in interleukin-2 concentrations. These findings support the conclusion that the personalized supplementation system produced measurable enhancements in physical performance and biochemical health indicators, validating the claimed methods of tailoring supplement protocols based on biological and qualitative input.

These results demonstrate the utility of the claimed invention in dynamically generating and adapting supplement recommendations, thereby addressing the limitations of generic over-the-counter supplementation strategies and illustrating the practical applicability and technical advantages of the invention in real-world settings.

In some embodiments, certain aspects of the techniques described above may be implemented by one or more processors of a processing system executing software. The software comprises one or more sets of executable instructions stored or otherwise tangibly embodied on a non-transitory computer readable storage medium. The software can include the instructions and certain data that, when executed by the one or more processors, manipulate the one or more processors to perform one or more aspects of the techniques described above. The non-transitory computer readable storage medium can include, for example, a magnetic or optical disk storage device, solid state storage devices such as Flash memory, a cache, random access memory (RAM) or other non-volatile memory device or devices, and the like. The executable instructions stored on the non-transitory computer readable storage medium may be in source code, assembly language code, object code, or other instruction format that is interpreted or otherwise executable by one or more processors.

A computer readable storage medium may include any storage medium, or combination of storage media, accessible by a computer system during use to provide instructions and/or data to the computer system. Such storage media can include, but is not limited to, optical media (e.g., compact disc (CD), digital versatile disc (DVD), Blu-Ray disc), magnetic media (e.g., floppy disc, magnetic tape, or magnetic hard drive), volatile memory (e.g., random access memory (RAM) or cache), non-volatile memory (e.g., read-only memory (ROM) or Flash memory), or microelectromechanical systems (MEMS)-based storage media. The computer readable storage medium may be embedded in the computing system (e.g., system RAM or ROM), fixedly attached to the computing system (e.g., a magnetic hard drive), removably attached to the computing system (e.g., an optical disc or Universal Serial Bus (USB)-based Flash memory), or coupled to the computer system via a wired or wireless network (e.g., network accessible storage (NAS)). Note that not all of the activities or elements described above in the general description are required, that a portion of a specific activity or device may not be required, and that one or more further activities may be performed, or elements included, in addition to those described. Still further, the order in which activities are listed are not necessarily the order in which they are performed. Also, the concepts have been described with reference to specific embodiments. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of the present disclosure.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims. Moreover, the particular embodiments disclosed above are illustrative only, as the disclosed subject matter may be modified and practiced in different but equivalent manners apparent to those skilled in the art having the benefit of the teachings herein. No limitations are intended to the details of construction or design herein shown, other than as described in the claims below. It is therefore evident that the particular embodiments disclosed above may be altered or modified and all such variations are considered within the scope of the disclosed subject matter. Accordingly, the protection sought herein is set forth in the claims below.

What is claimed is:

1. A computer-implemented method for generating a personalized nutritional supplement formulation for an athletic user, comprising:
receiving, by a processor, biological data associated with the user, the biological data comprising at least one of genetic data, blood metabolite levels, lipid profile data, or urine analysis data;
generating, by the processor, a digital user profile based on the received biological data;
receiving, by the processor, structured qualitative data including at least one of a user-entered dietary preference, a subjective health goal, or performance feedback;
processing, by a machine learning model, the biological data and the structured qualitative data to generate a supplement profile comprising one or more recommended ingredients and dosages;
generating, by the processor, a manufacturing instruction set based on the supplement profile; and
transmitting the manufacturing instruction set to a supplement compounding system configured to formulate a physical supplement based on the manufacturing instruction set.

2. The method of claim 1, further comprising validating the supplement profile using a performance optimization algorithm that compares predicted outcomes to target performance metrics.

3. The method of claim 1, further comprising storing the digital user profile in a secure database with auditable access logging.

4. The method of claim 1, wherein the machine learning model is configured to be trained on a dataset comprising previous supplement outcomes, athlete biometrics, and health response data.

5. The method of claim 1, further comprising monitoring a physiological response of the athletic user to the formulated supplement using wearable sensor data.

6. The method of claim 1, wherein the biological data is obtained via laboratory testing and automatically uploaded via an electronic health record interface.

7. The method of claim 1, wherein the structured qualitative data is collected through a user-facing application with guided question logic.

8. The method of claim 1, further comprising iteratively updating the supplement profile based on subsequent biological data and user feedback.

9. A system for personalized supplement formulation, comprising:
a user interface configured to receive input data including biological information and structured qualitative feedback;
a processor configured to generate a digital user profile based on the input data;
a supplement recommendation engine comprising a trained machine learning model configured to output a supplement formulation profile; and
a manufacturing instruction generator configured to generate and transmit manufacturing instructions to a supplement compounding device.

10. The system of claim 9, wherein the biological information includes at least one of genetic sequencing data, blood chemistry metrics, lipid panel results, or urinary metabolite levels.

11. The system of claim 9, wherein the structured qualitative feedback includes responses to performance evaluation prompts delivered via a mobile application.

12. The system of claim 9, wherein the user interface comprises a secure application that enforces data encryption and authentication protocols.

13. The system of claim 9, wherein the supplement recommendation engine is further configured to adjust ingredient ratios based on detected metabolic efficiency of the user.

14. The system of claim 9, further comprising a data storage module configured to store user profiles and supplement formulation histories with version tracking.

15. The system of claim 9, wherein the manufacturing instruction generator outputs a dosage, packaging specification, and allergen profile for a compoundable supplement unit.

16. A non-transitory computer-readable medium storing instructions that, when executed by a processor, cause the processor to perform a method comprising:
receiving genetic and metabolic testing results for a user;
generating a digital user profile;
processing the user profile and structured user feedback through a machine learning engine;
outputting a personalized nutritional supplement formulation; and
transmitting supplement compounding instructions to a manufacturing module.

17. The non-transitory computer-readable medium of claim 16, wherein the machine learning engine is trained using supervised learning based on historical user outcome data.

18. The non-transitory computer-readable medium of claim 16, wherein the structured user feedback comprises a self-reported energy level, workout performance rating, and dietary adherence log.

19. The non-transitory computer-readable medium of claim 16, further comprising updating the digital user profile based on longitudinal biomarker trends.

20. The non-transitory computer-readable medium of claim 16, wherein the supplement formulation includes at least one microencapsulated active ingredient tailored to user absorption efficiency.
